(19) United States
(12) Patent Application Publication
(10) Pub. No.: US 2022/0062130 A1
(43) Pub. Date: Mar. 3, 2022

(54) SKIN CARE COMPOSITIONS INCLUDING AZELAIC ACID AND METHODS OF MAKING A SKIN CARE COMPOSITION

(71) Applicant: L'OREAL, Paris (FR)

(72) Inventors: Alison Nicole TAYLOR, Jersey City, NJ (US); Patricia BRIEVA, Manalapan, NJ (US); Stephen Matthew LYNCH, Westfield, NJ (US); Maggie SU, Cranford, NJ (US)

(73) Assignee: L'OREAL, Paris (FR)

(21) Appl. No.: 17/004,940

(22) Filed: Aug. 27, 2020

(51) Int. Cl. A61K 8/362; A61K 8/06; A61Q 19/08; A61Q 19/02; A61Q 19/10; G06Q 30/06; G16H 50/30; G09B 5/02; A45D 44/00; B01F 13/10

(52) U.S. Cl. CPC A61K 8/362; G06F 3/14; A61Q 19/08; A61Q 19/02; A61Q 19/10; G06Q 30/0621; G06Q 30/0643; G16H 50/30; G09B 5/02; A45D 44/00; B01F 13/1058; B01F 13/1063; A61K 2800/30; A45D 2044/007; B01F 2215/0031; A61K 8/062

(57) ABSTRACT

A skin care formulation includes azelaic acid. The skin care formulation also includes a first booster composition including at least one first active booster ingredient. The skin care formulation further includes a second booster composition including at least one second active booster ingredient. The skin care formulation is a water-based emulsion that is stable for at least 3 months. A method of forming a skin care formulation includes combining an anti-acne booster composition, a first booster composition, a second booster composition, and an aqueous base composition to form the skin care formulation. The anti-acne booster composition includes azelaic acid.

### FIELD OF TECHNOLOGY

The present disclosure is directed to systems, devices, and methods including a skin care composition and a treatment skin conditions. More specifically, the present disclosure is directed to systems, devices, and methods including a skin care composition that has targeted active ingredients including azelaic acid for treating target skin conditions and methods for creating customized skin care compositions.

### BACKGROUND

The Ideal Renewing Ampoule from CellCure contains low levels of hydroxyethyl urea, tranexamic acid, and azelaic acid.

The Metamorphosis Softening Serum and Enlightening Lotion from JolieMD contains licorice root extract and low levels of azelaic acid. This product is a kit that contains a softening serum including azelaic acid and licorice root extract and an enlightening lotion including licorice root extract.

The Blue Algae Vitamin C Dark Spot Correcting Peel from Algenist contains low levels of tranexamic acid and azelaic acid and is substantially anhydrous.

Personalized skin care systems are desirable for consumers to provide a skin care composition that meets the skin care needs of the individual consumer.

Examples of personalized product lines include MaCrèmeSurMesure by Dr. Pierre Ricaud, Codage Paris, Customized TAM by Yue Sai, and prescription personalized serums (Re-Plasty) by Helena Rubinstein.

Helena Rubinstein uses a skinprofiler, a cutaneous analysis device that generates quantitative data to analyze a variety of skin attributes to diagnose consumers in-store and provide them with a personalized prescription product consisting of one universal serum base and single dose of concentrate. Helena Rubenstein products only consist of one single dose of concentrate containing actives that seek to address all skincare needs. The Helena Rubinstein skinprofiler measures elasticity, pigmentation, and texture of the skin and prescribes concentrate based on these results only.

Codage Paris has a custom-made skincare product line. Consumers use a website diagnostic tool in which they answer 23 questions that were developed by different specialists (including dermatologists, pharmacists, and nutritionists). The Codage Paris system does not include a base composition with personalized boosters. A diagnostic tool utilized for Codage is an online survey which automatically ‘prescribes’ a final product based on provided answers. The Codage Product provides a final pre-prepared formula for the consumer, which is sent directly from store to consumer, where no mixing is required.

MaCrèmeSurMesure by Dr. Pierre Ricaud similarly uses an online diagnostic tool in which consumers are given a questionnaire on their current skin and lifestyle. Based on the answers they provide, the website provides them with a cream base and combination of three active concentrates. Each “concentrate” for the MaCrèmeSurMesure only contains a single active ingredient. Typical “concentrates” for the MaCrèmeSurMesure products consist solely of pure extracts (100% active extract of chufa tubers, for example) and do not provide formulations. The consumer is sent a kit with their products and mixes at home with a small mixing tool provided, which is not easily co-soluble.

Yue Sai is a Customized TCM Beauty Solution Ultimate Refining Serum that includes in-store formulations in China. The tailoring of the formulations were limited and included only aqueous based compositions.

There remains a need to provide an effective, skin care composition and method for treating skin that is customized to specific, individual customer target skin compositions and that is stable and efficacious.

### BRIEF SUMMARY

In an exemplary embodiment, a skin care formulation includes at least three active ingredients, at least one of which is an anti-acne booster comprising azelaic acid. In various embodiments, the at least one anti-acne booster includes 1-20% by weight azelaic acid. In some embodiments, the skin care formulation includes 1-15% by weight azelaic acid, or 3-15% by weight azelaic acid, and in some particular embodiments, the skin care formulation includes about 3%, or about 5%, or about 10%, or about 15% by weight azelaic acid. The skin care formulation also includes a first booster composition including at least one first active booster ingredient. The skin care formulation further includes a second booster composition including at least one second active booster ingredient. The skin care formulation is a water-based emulsion that is stable for at least 3 months.

In another exemplary embodiment, a method of forming a skin care formulation includes combining an anti-acne booster composition, a first booster composition, a second booster composition, and an aqueous base composition to form the skin care formulation. The anti-acne booster composition includes azelaic acid. The first booster composition includes at least one first active booster ingredient. The second booster composition includes at least one second active booster ingredient. In some embodiments, the anti-acne booster composition includes azelaic acid from about 1-30%, and in some embodiments from about 1-25%, and in some embodiments from about 20-25%, or at least about 20%, or about 22-24%. In some embodiments, the skin care formulation formed according to the method includes 1-20% by weight azelaic acid together with the additional first and second active booster ingredients. In other embodiments, the skin care formulation includes 1-15% by weight azelaic acid, or 3-15% by weight azelaic acid, and in some particular embodiments, the skin care formulation includes about 3%, or about 5%, or about 10%, or about 15% by weight azelaic acid. The skin care formulation is a water-based emulsion that is stable for at least 3 months.

In yet another exemplary embodiment, a method manages a skin care system for providing a customized skin care product to a user, the skin care system including one or more input/output devices configured to display information to a user and receive input from the user, one or more cosmetic dispensing systems configured to dispense and mix a cosmetic composition, and a skin care management system, coupled to the one or more input/output devices and the one or more cosmetic dispensing systems, and including a microprocessor and a memory. The method includes displaying, by the skin care management system, one or more visual skin guides to the user, each of the visual skin guides corresponding to one or more skin conditions including one or more visual representations of the one or more skin conditions. The method also includes receiving, by the skin care management system, one or more inputs from the user including skin information corresponding to the one or more visual representations of the one or more skin conditions of the one or more visual skin guides. The method further includes determining, by the skin care management system, one or more skin conditions based on the one or more inputs from the user, where the one or more skin conditions comprises acne. The method yet further includes determining, by the skin care management system, one or more severity scores based on the one or more skin conditions. The method also includes determining, by the skin care management system, a primary skin condition based on the severity scores.

The method further includes selecting, by the skin care management system, a first ingredient based on the one or more severity scores. The method yet further includes generating, by the skin care management system, a compatibility profile based on the first ingredient. The method also includes determining, by the skin care management system, one or more secondary skin conditions based on the one or more severity scores. The method further includes selecting, by the skin care management system, an additional ingredient based on the one or more severity scores. The method yet further includes updating, by the skin care management system, the compatibility profile based on the additional ingredient. The method also includes displaying, by the skin care management system, one or more cosmetic formulations to the user. The method further includes receiving, by the skin care management system, a selection of one or more cosmetic formulations from the user. The cosmetic formulation includes a base composition, an anti-acne booster composition including the azelaic acid, where the first ingredient comprises from about 1-30%, and in some embodiments from about 1-25%, and in some embodiments from about 20-25%, or at least about 20%, or about 22-24%. In some embodiments, the skin care formulation formed according to the method includes 1-20% by weight azelaic acid. In other embodiments, the skin care formulation includes 1-15% by weight azelaic acid, or 3-15% by weight azelaic acid, and in some particular embodiments, the skin care formulation includes about 3%, or about 5%, or about 10%, or about 15% by weight azelaic acid. The cosmetic formulation also includes a first booster composition including at least one first active booster ingredient, and a second booster composition including at least one second active booster ingredient.

Other features and advantages of the present disclosure will be apparent from the following more detailed description of the preferred embodiment which illustrates, by way of example, the principles of the disclosure.

### BRIEF DESCRIPTION OF THE DRAWINGS

FIG. 1 shows a cosmetic dispensing system, according to an embodiment of the present disclosure.

Wherever possible, the same reference numbers will be used throughout the drawings to represent the same parts.

### DETAILED DESCRIPTION

A booster composition, according to the present disclosure, includes azelaic acid as an active ingredient. In some embodiments, the booster composition is used in a skin care formulation. In some embodiments, the booster composition is used in a skin care formulation dispensing system. In some embodiments, the booster composition is used in a skin care management system.

A skin care formulation, according to the present disclosure, includes formulation ingredients of a base composition, a booster composition including azelaic acid, and at least two additional booster compositions.

A method and system, according to the present disclosure, produces a targeted and efficacious product from a comprehensive skin condition diagnosis practice. The skin conditions of a consumer are analyzed either automatically, using a digital system or manually, for treatment with a skin care product. In one embodiment, consumers are provided with a questionnaire or shown representative photos of common skin conditions, where the consumers/patients provide information correlating to their individual skin care needs. In one embodiment, a skin care professional fills out a questionnaire based upon information from or about the consumer. Based on the correlation to the skin information obtained, the consumers are provided with a formulation including a base composition and boosters having actives, which form a stable composition having efficacious concentrations of active ingredients corresponding to the desired skin treatment.

Formulation Ingredients

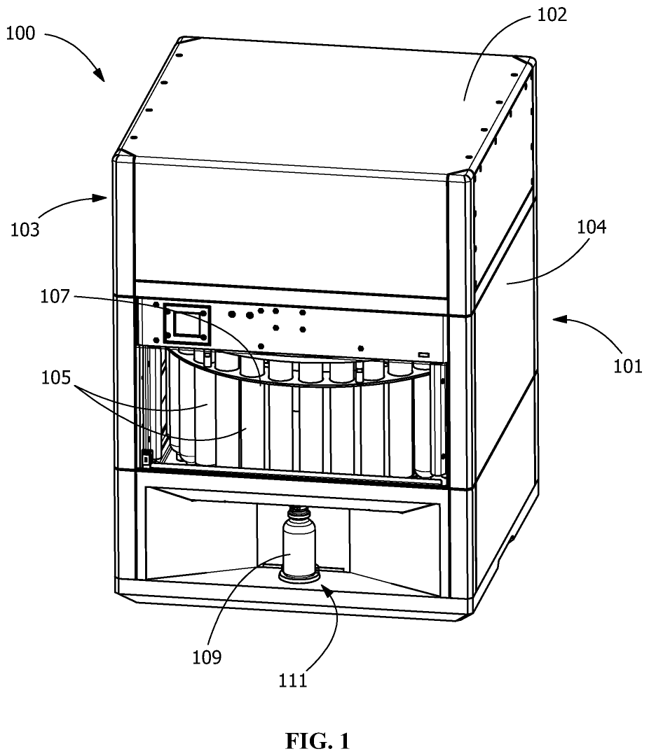

In one embodiment, the formulation ingredients include a first ingredient, a second ingredient, a third ingredient, and a fourth ingredient that correspond to a base composition, a booster composition including azelaic acid, and two additional booster compositions. In this embodiment, each of the first ingredient, the second ingredient, the third ingredient, and the fourth ingredient are independently selected from an aqueous emulsion base composition and a booster composition selected from the group consisting of a first grade exfoliating agent; a second grade exfoliating agent, the second grade exfoliating agent providing greater exfoliating than the first grade exfoliating agent; a first grade whitening agent; a second grade whitening agent, the second grade whitening agent providing greater whitening than the first grade whitening agent; a first grade anti-aging agent; and a second grade anti-aging agent, the second grade anti-aging agent providing greater anti-aging effects than the first grade anti-aging agent. In embodiments where the formulation does not include azelaic acid, the base composition may alternatively be an aqueous alcohol composition. The skin care composition formed from the formulation is stable and has efficacious concentrations of active ingredients corresponding to skin conditions of a consumer. Each of the ingredients is preferably added from a booster or base reservoir. In one embodiment, as shown in FIG. 1, booster compositions and base compositions are contained in dispensing dosing receptacles 105.

The formulation ingredients, according to the present disclosure, include a booster composition that includes azelaic acid as an active ingredient. In some embodiments, the booster is an anti-acne booster. In one embodiment, the azelaic acid anti-acne booster is formulated to work with an exfoliation booster composition, a whitening booster composition, and/or an anti-aging booster composition.

In some embodiments, the skin care formulation is essentially free of oils other than silicone oils. In other embodiments, the skin care formulation may contain one or more oils. For example, formulations including retinol may include soybean oil.

Azelaic Acid Anti-Acne Booster Composition

In one embodiment, the azelaic acid anti-acne booster is a highly concentrated formulation, designed to operate in conjunction with a skin care customization machine and software. The azelaic acid anti-acne booster includes azelaic acid from about 1-30%, and in some embodiments from about 1-25%, and in some embodiments from about 20-25%, or at least about 20%, or about 22-24%, dispersed in an aqueous dispersion that is essentially free of one or more of: alcohols, oils other than silicone oils, and combinations thereof. The azelaic acid anti-acne booster may be targeted for oily, acne-prone skin, with a goal being to reduce the appearance of active lesions, post-inflammatory hyperpigmentation, post-inflammatory erythema, and redness.

In one embodiment, the azelaic acid anti-acne booster is formulated to operate effectively in a dispensing machine without causing undue issues, such as leaking or dripping. The azelaic acid is uniformly dispersed and suspended within the booster to ensure consistent delivery of actives and to prevent clogging. The azelaic acid anti-acne booster is combined with an aqueous emulsion base composition and two other booster compositions to create a final formulation. After the formulation ingredients are dispensed, the final formulation is mixed and provided to the consumer. In some embodiments, the skin care formulation formed using the anti-acne booster includes 1-20% by weight azelaic acid. In other embodiments, the skin care formulation includes 1-15% by weight azelaic acid, or 3-15% by weight azelaic acid, and in some particular embodiments, the skin care formulation includes about 3%, or about 5%, or about 10%, or about 15% by weight azelaic acid. Thus, the concentration of azelaic acid in the formulation may include, by weight about 1% to about 20%, about 1% to about 15%, about 5% to about 12%, about 1% to about 5%, about 3% to about 7%, about 5% to about 12%, about 10% to about 15%, about 5%, about 10%, about 12%, about 15%, not more than about 5%, not more than about 12%, about 3% to about 15% or any value, range, or sub-range therebetween. The concentration of azelaic acid is determined by a professional, based on a questionnaire completed for the consumer, or at the determination of the professional.

In one embodiment, the azelaic acid anti-acne booster includes at least 50% water by weight.

In one embodiment, the azelaic acid anti-acne booster includes up to about 20% of one or more glycols by weight, alternatively at least 2%, alternatively at least 3%, alternatively at least 5%, alternatively 5% to 15%, alternatively 5% to 25%, alternatively at least 15%, alternatively 15% to 25%, alternatively about 20% to about 21%, or any value, range, or sub-range therebetween.

In one embodiment, the azelaic acid anti-acne booster includes less than 5% alcohol by weight, alternatively about 4%, alternatively about 4% or less, alternatively 3% or less, alternatively 1% or less, or any value, range, or sub-range therebetween. In one embodiment, the azelaic acid anti-acne booster is free or essentially free of alcohol. For purposes hereof, free or essentially free means that any monoalcohol agent is present in an amount that is at or below 5%, or 4%, or 3%, or 2% or 1% of alcohol, based on the total weight of the composition, and thus, in some embodiments, the composition is free or essentially free of alcohols comprising one or more of monoalcohols such as monohydric C₁-C₈ alcohols such as ethanol, propanol, butanol, isopropanol, isobutanol, and benzyl alcohol, and phenylethyl alcohol.

In one embodiment, the azelaic acid anti-acne booster includes one or more silicone oils. Appropriate silicone oils may include, but are not limited to, dimethicone, dimethiconol, silicone crosspolymers, and/or polysiloxanes. In one embodiment, any silicone oil may be included.

In one embodiment, the azelaic acid anti-acne booster includes one or more polymers. Appropriate polymers may include xanthan gum, dimethicone, dimethiconol, silicone polymers, acrylate crosspolymers, non-ionic polymers, and/or anionic polymers.

In one embodiment, the azelaic acid anti-acne booster is formulated to work with other boosters containing a high concentration of active ingredients, such as, for example, hydroxyethyl urea, hydroxyethyl piperazine ethanesulfonic acid, tranexamic acid, licorice root extract, mulberry extract, and/or retinol up to 0.5%.

In one embodiment, the azelaic acid anti-acne booster is not combined with any other booster that would cause the azelaic acid to become unstable in the skin care formulation, such as, for example, certain acids. In one embodiment, the azelaic acid anti-acne booster is not combined with a booster containing a second grade exfoliating agent of glycolic acid, lactic acid, and sodium phytate. In one embodiment, the azelaic acid anti-acne booster is not combined with a booster containing a second grade whitening agent of phenylethyl resorcinol. In one embodiment, the azelaic acid anti-acne booster is not combined with an aqueous alcohol base composition.

In one embodiment, the azelaic acid anti-acne booster is stable for at least 27 months as packaged prior to opening. In one embodiment, the azelaic acid anti-acne booster is stable for at least 6 month after exposure upon opening.

Exfoliation Booster Composition

In one embodiment, the ingredient selected may be an ingredient that provides exfoliation to improve skin appearance. As noted above, the selection of the ingredient may include a response to the consumer's skin condition, the previously selected ingredients, the compatibility profile of the already selected ingredients, and the identity of the previously selected ingredients. Active ingredients corresponding to exfoliation include one or more actives that provide exfoliation of the skin. The exfoliation booster composition includes a first grade exfoliating agent or a second grade exfoliating agent. The second grade exfoliating agent provides greater exfoliation than the first grade exfoliating agent. In the formulation and the selection of the individual ingredients, the first grade exfoliating agent and the second grade exfoliating agent are not the same.

For example, in one embodiment, the actives corresponding to an ingredient corresponding to a first grade exfoliating agent or a second grade exfoliating agent include (except in the case of an incompatibility with a base composition or one or more other booster compositions) one or more of glycolic acid, lactic acid, sodium phytate, 4-(2-hydroxyethyl)-1-piperazineethanesulfonic acid (HEPES), hydroxyethyl urea, salicylic acid, citric acid, capryloyl salicylic acid, and other components that provide improvement to skin appearance, and combinations thereof. Efficacious concentrations of the ingredients include concentration of active ingredients sufficiently high to provide exfoliation. For example, efficacious concentrations of from 1 wt % to about 15 wt % of glycolic acid, lactic acid, and sodium phytate; from 0.5 wt % to about 5 wt % 4-(2-hydroxyethyl)-1-piperazineethanesulfonic acid; from 2 wt % to about 10 wt % hydroxyethyl urea; from 0.05 wt % to about 0.04 wt % salicylic acid, from 1 wt % to about 15 wt % citric acid; and from 0.01 wt % to about 0.5 wt % capryloyl salicylic acid. In one embodiment, the active ingredients in a booster composition corresponding to the first grade exfoliating agent include 6% 4-(2-hydroxyethyl)-1-piperazineethanesulfonic acid and 5.45% hydroxyethyl urea by weight of the skin care composition. In one embodiment, the active ingredients in a booster composition corresponding to the second grade exfoliating agent include 6.7% glycolic acid, 3.3% lactic acid and 1.7% sodium phytate by weight of the skin care composition.

Whitening Booster Composition

In one embodiment, the ingredient selected may be an ingredient that provides skin whiteness. As noted above, the selection of the ingredient may include a response to the consumer's skin condition, the previously selected ingredients, the compatibility profile of the already selected ingredients and the identity of the previously selected ingredients. Active ingredients corresponding to skin whiteness include one or more actives that provide improvement to skin whiteness. The whitening booster composition includes a first grade whitening agent or a second grade whitening agent. The second grade whitening agent provides greater whitening than the first grade whitening agent. In the formulation and the selection of the individual ingredients, the first grade whitening agent and the second grade whitening agent are not the same.

For example, in one embodiment, the actives corresponding to an ingredient corresponding to a first grade whitening agent or a second grade whitening agent include one or more of niacinamide, kojic acid, licorice extract, mulberry extract, tranexamic acid, urea, phenylethyl resorcinol, ascorbic acid, and other components that provide improvement to skin whiteness, any other suitable soluble/dispersible targeted active ingredient, and combinations thereof. Efficacious concentrations of the ingredients include concentration of active ingredients sufficiently high to provide whitening. For example, efficacious concentrations of from 0.01 wt % to about 2 wt % of licorice extract; from 0.001 wt % to about 0.5 wt % mulberry extract; from 0.25 wt % to about 10 wt % niacinamide; from 0.5 wt % to about 5 wt % kojic acid; from 0.01 wt % to about 1 wt % phenylethyl resorcinol; from about 0.1 wt % to about 4 wt % tranexamic acid; and from about 1 wt % to about 15 wt % ascorbic acid. In one embodiment, the active ingredients in the first grade whitening agent include 0.1% licorice extract; and 0.0025% mulberry extract, by weight of the skin care composition. In one embodiment, the active ingredients in the second grade whitening agent include 3% niacinamide and 1% kojic acid by weight of the skin care composition. In one embodiment, the active ingredients in the second grade whitening agent include 0.5% phenylethyl resorcinol by weight of the skin care composition.

Anti-Aging Booster Composition

In one embodiment, the ingredient selected may be an ingredient that provides anti-aging. As noted above, the selection of the ingredient may include a response to the consumer's skin condition, the previously selected ingredients, the compatibility profile of the already selected ingredients and the identity of the previously selected ingredients. Active ingredients corresponding to anti-aging include one or more actives that provide improvement to skin appearance. The whitening booster composition includes a first grade anti-aging agent or a second grade anti-aging agent. The second grade anti-aging agent provides greater anti-aging effects than the first grade anti-aging agent. In the formulation and the selection of the individual ingredients, the first grade anti-aging agent and the second grade anti-aging agent are not the same.

For example, in one embodiment, the actives corresponding to an ingredient corresponding to a first grade anti-aging agent or a second grade anti-aging agent include one or more of C-beta-D-xylopyranoside-2-hydroxypropane (Pro-Xylane), retinol, peptides, caffeine, and other components that provide improvement to skin texture, any other suitable soluble/dispersible targeted active ingredient, and combinations thereof. Efficacious concentrations of the ingredients include concentration of active ingredients sufficiently high to provide anti-aging. For example, efficacious concentrations of from 0.5 wt % to about 10 wt % of C-beta-D-xylopyranoside-2-hydroxypropane; from 0.1 wt % to about 1.1 retinol; from 0.5 wt % to about 10 wt % ascorbic acid; from 0.1 wt % to about 1 wt % hyaluronic acid; from 0.01 wt % to about 1 wt % peptides; and from 0.1 wt % to about 1 wt % caffeine. In one embodiment, the active ingredients in the first grade anti-aging agent include 3.5% C-beta-D-xylopyranoside-2-hydroxypropane, by weight of the skin care composition. In one embodiment, the active ingredients in the second grade anti-aging agent include 0.1% or 0.3% or 0.5% or 1.0% retinol, by weight of the anti-aging booster composition.

Booster Additives

The booster compositions noted above may further include additional additives. For example, the booster compositions may include humectants, such as glycol and glycerin, soothing ingredients, essential oils, Vitamin E, emollients or other ingredients for skin enhancement, solubilization or other beneficial or efficacious purpose.

In one embodiment, the booster compositions are contained in dispensing dosing receptacles 105 (see FIG. 1). The concentrations of the ingredients for the booster ingredients (i.e., the exfoliating agents, the whitening agents and the anti-aging agents) in the dispensing dosing receptacles 105 is preferably at or near the solubility limits of the individual ingredients.

Base Composition

In addition to the plurality of booster compositions, one or more base compositions are also provided.

Aqueous Alcohol Base Composition

One base composition suitable for use in the skin care composition includes an aqueous alcohol base composition. The aqueous composition includes an aqueous composition, comprising, consisting essentially of or consisting of an alcohol, such as ethanol or denatured alcohol. In one embodiment, the base composition includes an aqueous alcohol base composition comprising about 35 wt % alcohol, balance water. In one embodiment, an aqueous alcohol base composition is not used with boosters including azelaic acid.

Aqueous Emulsion Base Composition

One base composition suitable for use in the skin care composition includes an aqueous emulsion base composition. The aqueous emulsion base composition includes an aqueous composition, comprising, consisting essentially of or consisting of an emulsifier, such as polyacrylate crosspolymer-6. In one embodiment, the base composition includes an aqueous emulsion base composition comprising about 0.6 wt % polyacrylate crosspolymer-6.

The base compositions may include components suitable for providing skin care benefits, such as moisturization, protection of skin barrier, or other skin benefit.

In one embodiment, the base compositions are contained in dispensing dosing receptacles 105 (see FIG. 1). The concentration of the ingredients for the base compositions in the dispensing dosing receptacles 105 is preferably at the above indicated concentrations, where the base compositions include ranges of ingredients that permit dilution of the booster compositions to their efficacious concentrations, while maintaining the benefits of the booster composition.

Auxiliaries

In an embodiment, the composition of the disclosure may also contain adjuvants that are common in cosmetics, such as humectants, preserving agents, antioxidants, complexing agents, solvents, fragrances, bactericides, odor absorbers, vitamins, moisturizers, self-tanning compounds, and other active agents. The amounts of these various adjuvants are those conventionally used in the field under consideration, for example, from 0.01% to 20% of the total weight of the composition. In one embodiment, the additives or adjuvants would be added to the booster formulations to functionalize them for specific targeted treatments/needs.

Of course, a person skilled in the art will take care to choose this or these optional additional compounds so that the advantageous properties intrinsically attached to the composition, in accordance with the disclosure, are not, or not substantially, detrimentally affected by the envisaged addition or additions.

Needless to say, a person skilled in the art will take care to select this or of these optional additional compound(s), and/or the amount thereof, such that the advantageous properties of the composition, according to the disclosure, are not, or are not substantially, adversely affected by the envisaged addition.

Cosmetic Dispensing System

FIG. 1 shows a cosmetic dispensing system 100, according to an embodiment of the present disclosure. The cosmetic dispensing system 100 includes a dispensing module 101 and a mixing module 103. A hinged lid 102 is hingeably attached to the outer housing 104. Dispensing module 101 provides a plurality of dispensing dosing receptacles 105 mounted on a carousel 107 that is rotatably driven to align the dispensing dosing receptacles 105 with a receiving receptacle 109 positioned in a receiving portion 111 of the cosmetic dispensing system 100. The carousel 107 and dispensing dosing receptacles 105 are driven such that, when a formula is provided, the dispensing dosing receptacles 105 are independently and sequentially aligned with the receiving portion 111 to provide ingredients from the dispensing dosing receptacles 105 to the receiving receptacle 109 to provide a skin care composition having a desired ingredient combination according to the formulation corresponding to the consumer skin condition and the compatibility profile between the base compositions and booster compositions in the formulation.

Method

In one embodiment, a method includes obtaining skin information corresponding to a consumer. The method further includes generating a skin condition corresponding to the skin information. Exemplary skin conditions for the first skin condition may include, but are not limited to, skin requiring displaying some level of acne, oiliness/dryness of the skin, requiring exfoliation (e.g., sensitivity), requiring lightening/whitening, and/or requiring anti-aging. One example may include a very dry skin condition and the utilization of base without drying components but containing nourishing attributes. Furthermore, a very oily skin condition may utilize a hydroalcoholic base composition to help resolve this skin concern. The skin condition determined may also include a comparison of the numerical value, grade or other value indicator for the severity of the skin condition to determine which are suitable for treatment. In one particularly suitable embodiment, the correlation may include a consultation with a skin care professional, who may assist the consumer in determining the priority in which skin conditions may be present and/or desirably treated or may assist in comparing the severity score in determining the target skin conditions. For example, the skin condition may include, in one embodiment, a severity score to determine specific exfoliants, lightening agents, and/or anti-aging agents that correspond to the severity of the skin condition. The ingredients selected are dependent upon the skin condition. In one embodiment, the base composition is determined by the patient's skin type. For example, in another embodiment, the skin condition may include, a severity score to determine dosing or grade of exfoliating agents, lightening agents, and/or anti-aging agents that correspond to the severity of the skin condition or the priority of which the skin conditions should be treated. In one embodiment, the skin condition that is first utilized to select an ingredient includes a skin condition that corresponds to the skin condition of the consumer that has the greatest severity. In another embodiment, the skin condition utilized to select an ingredient may include a skin condition that relates to a condition that the consumer or skin care professional prioritizes as a greater need for treatment.

The skin condition may determine scores for categories of concern, such as, for example: pigmentation concern, sensitivity, complexion, wrinkles, texture, hydration, or oily/dry skin. In one particularly suitable embodiment, consultation from a skin care professional may be utilized. The skin care consultant may provide direction relating to the consumer's current regimen, skin type, skin concerns and lifestyle. To assess top skin concerns, diagnostic tools which may include, but are not limited to, a combination of a special prescription card (ranking of concerns) and reference pictures (skin atlas) are utilized. In addition, the skin information and the skin condition may be determined utilizing automated systems. For example, different devices for performing the skin diagnosis are readily understood in the art, such as the Lancome Diagnos ABS, HR Skinscope, Biotherm Bluesmart, Kiehl's Skinprofiler V.0, C A Dermanalyzer, and the Vichy Vichyconsult.

In one embodiment, determining the skin information corresponding to a consumer is done via a visual display shown to the consumer. Suitable types of visual displays include, but are not limited to, books, brochures and pamphlets including, but not limited to, pictures and/or description of skin condition and scale, clinical pictures, and photos taken with instrumental devices. The pictures and/or photos may include representations of skin conditions for panelists of different ethnicities and skin types. The visual display will correspond to the varying ethnicities and skin types. A selection of a specific visual display is made to consumer's ethnicity and/or skin types. The visual skin guide is a device including visual representations, including, but not limited to, images, pictorial representations or similar visual devices that provide information regarding categorical skin conditions. In one embodiment, each of the visual skin guides includes a plurality of visual representations of a categorical skin condition. In this embodiment, each of the visual representations corresponds to a severity score for the categorical skin condition on the visual skin guide. As noted above, the severity score may be utilized to determine which type of ingredient may be added to the formulation or which grade of ingredient may be added to the formulation.

In one embodiment, an information processing apparatus is configured to output a series of questions to the user to collect information about the consumer for collecting skin information. However, alternative embodiments are also available in which the user enters information directly into appropriate fields displayed on the information processing apparatus without being prompted by displayed questions in order to input the information into the information processing apparatus.

In one embodiment, the method further includes selecting an ingredient corresponding to the skin condition. The ingredient includes a base composition or a booster composition that corresponds to the skin condition. In one embodiment, the booster composition includes azelaic acid. The selection of ingredients includes consideration of the previously selected ingredients and the compatibility profile of the previously selected ingredient. In addition, the selection of ingredient includes correlation of the skin condition to a specific ingredient or type of ingredient (e.g., anti-acne agents, exfoliating agents, whitening agents and/or anti-aging agents). Alternatively, the selection of ingredient may include a prioritization based upon other factors, such as consumer preference, or order of treatment from a skin care professional. In the case of the ingredient, the primary consideration of the selection of the ingredient is the skin condition, and its severity score or priority. In correlating the skin condition, the severity or priority includes a determination of the grade of ingredient.

In one embodiment, after the ingredient is determined, a compatibility profile is determined corresponding to the previously selected ingredients. The compatibility profile includes an inclusion or exclusion of additional ingredients to the formulation. That is, the compatibility profile provides acceptable combinations of booster composition and base composition combinations made with the previously selected ingredients that forms stable and efficacious skin care compositions. Compatibility for use in the compatibility profile includes, but is not limited to, combinations of base compositions and booster compositions that have 1) stability, including phase compatibility and solubility, 2) desirable pH, including a pH that does not result in esterification or similar incompatibility effects and 3) efficacy, including combinations of base compositions and booster compositions that retain the efficacy to provide the desired treatment for the skin condition determined in the formulation. “Stable”, “Stability” and grammatical variations thereof, indicate a macroscopic homogeneous substance that does not have variation in texture or varying phases physically noticeable. In addition, microscopically the emulsion of the composition has the appearance of being homogeneous with the droplets and no dynamic changes in the microscope image. Examples of compatibility that result in a stable efficacious skin care composition include ingredients that have compatible pH, such that the combination of the ingredients do not provide esterification, crystallization, separation, and precipitation or other incompatible effects. In addition, compatibility may include ingredients that have compatible solubility that result in a stable emulsion, where the ingredients do not readily break into phases. In addition, compatibility may include ingredients that retain efficacy of individual ingredients when combined with the other ingredients remain present in the formula over time periods and exposures to varying temperature. For example, as an incompatible combination, azelaic acid is not compatible with an exfoliation booster including 6.7% glycolic acid, 3.3% lactic acid, and 1.7% sodium phytate, because the combination results in an incompatible pH and results in separation of phases or precipitation of one or more active ingredients.

In one embodiment, the method further includes repeating the generating skin conditions, selecting the ingredient and generating the compatibility profile until a sufficient number of ingredients are selected to provide a formulation. The sufficient number of ingredients includes a number of ingredients that address the skin concerns of the consumer based upon the skin information or include a number of ingredients corresponding to a skin care plan or regiment. For example, the skin care plan may include ingredients for treatment of the top three or four skin conditions, which correspond to three for four ingredients. However, the present disclosure is not so limited, as any number of skin conditions may be treated. In one embodiment, the skin care plan would correspond to at least four ingredients, where the ingredients include a base composition, an azelaic acid booster composition, and at least two additional booster compositions, the base composition and the booster compositions corresponding to skin conditions of the consumer for treatment.

Once the desired number of ingredients are selected, a formulation including the ingredients is generated. In one embodiment, the formulation includes a base composition and at least two booster compositions. In another embodiment, the formulation includes a base composition and three booster compositions. In one embodiment, one of the booster compositions including azelaic acid. Once the formulation is generated, the formulation, including the selected ingredients, is dispensed. The dispensing may be done manually or via an automated dispenser. After or during the dispensing the dispensed formulation may be mixed. Mixing may be mixed manually or automatically during or after the dispensing. For example, in one embodiment, as show in FIG. 1, the mixing may be provided by the mixing module 103 of cosmetic dispensing system 100. In this embodiment, the receiving receptacle 109 is filled by the dispensing module 101 and then inserted into the mixing module 103, where the receiving receptacle is mixed to provide a stable, homogeneous mixture.

In an exemplary embodiment, a method includes obtaining skin information corresponding to a consumer. The method also includes generating a first skin condition corresponding to the skin information. The method further includes selecting a first ingredient corresponding to the first skin condition. The first ingredient includes a base composition or a booster composition that corresponds to the first skin condition.

After the first ingredient is determined, a first compatibility profile is determined corresponding to the previously selected ingredients. The first compatibility profile includes an inclusion or exclusion of additional ingredients to the formulation. That is, the compatibility profile provides acceptable combinations of booster composition and base composition combinations made with the first ingredient that forms stable and efficacious skin care compositions.

The method further includes generating a second skin condition corresponding to the skin information. The second skin condition also preferably corresponds to the first skin condition. The second skin condition may include a severity or priority corresponding to the skin information or particular concerns of the consumer.

The method further includes selecting a second ingredient corresponding to the second skin condition. The selection of a second ingredient includes consideration of the previous selected ingredients and the compatibility profile of the previously selected ingredient. That is, the second ingredient selected includes a base composition or a booster composition that corresponds to the second skin condition that is not the first ingredient. In addition, the selection of a second ingredient includes correlation of the skin condition, including a prioritization. In correlating the skin condition to the second ingredient, the severity or priority includes a determination of the grade of ingredient. Accordingly, a second ingredient is selected, which includes a first or second grade of ingredient.

After the second ingredient is determined, the method further includes generating a second compatibility profile corresponding to the second ingredient and the first ingredient. Like the first compatibility profile, the second compatibility profile includes an inclusion or exclusion of additional ingredients to the formulation. That is, the second compatibility profile provides acceptable combinations of booster composition and base composition combinations made with the first ingredient and the second ingredient that form stable and efficacious skin care compositions.

The method further includes generating a third skin condition corresponding to the skin information. The third skin condition also preferably corresponds to the first skin condition and the second skin condition. As noted above with respect to the first condition, the third skin condition may include a severity or priority corresponding to the skin information or particular concerns of the consumer.

The method further includes selecting a third ingredient corresponding to the third skin condition. The selection of a third ingredient includes consideration of the previously selected ingredients and the compatibility profile of the previously selected ingredient. That is, the third ingredient includes a base composition or a booster composition that corresponds to the third skin condition that is not the first ingredient or the second ingredient. In addition, the selection of a third ingredient includes correlation of the skin condition, including a prioritization. In correlating the skin condition to the second ingredient, the severity or priority includes a determination of the grade of ingredient. Accordingly, a third ingredient is selected, which includes a first or second grade of ingredient.

After the third ingredient is determined, the method further includes generating a third compatibility profile corresponding to the third ingredient, the second ingredient and the first ingredient. Like the first compatibility profile and the second compatibility profile, the third compatibility profile includes an inclusion or exclusion of additional ingredients to the formulation. That is, the third compatibility profile provides acceptable combinations of booster composition and base composition combinations made with the first ingredient, the second ingredient and the third ingredient that form stable and efficacious skin care compositions.

The method may further include generating a fourth skin condition corresponding to the skin information. The fourth skin condition also preferably corresponds to the first skin condition, the second skin condition and the third skin condition. As noted above with respect to the first condition, the fourth skin condition may include a severity or priority corresponding to the skin information or particular concerns of the consumer.

The method may further include selecting a fourth ingredient corresponding to the fourth skin condition. The fourth ingredient includes a base composition or a booster composition that corresponds to the fourth skin condition that is not the first ingredient, the second ingredient or the third ingredient.

A formulation including the first ingredient, the second ingredient, the third ingredient, and the fourth ingredient is generated. The formulation includes a first ingredient, a second ingredient, a third ingredient, and a fourth ingredient that corresponds to a base composition and three booster compositions. Once the formulation is generated and communicated, the first ingredient, the second ingredient, the third ingredient, and fourth ingredient are dispensed with a cosmetic dispensing system. The dispensing may be done manually or via an automated dispenser. In addition, after or during the dispensing the formulation may be mixed. The mixing may be mixed manually or automatically during or after the dispensing. For example, in one embodiment, as show in FIG. 1, the mixing may be provided by the mixing module 103 of cosmetic dispensing system 100.

According to the foregoing method, at least one of the skin conditions comprises acne and one of the generated ingredients is a booster comprising azelaic acid.

In one embodiment, a process or algorithm performed by the circuitry of an information processing apparatus selects the ingredients and determines the compatibility profile.

While the above has been described with respect to a first, second, third and fourth ingredient, other numbers of ingredients may be utilized, where additional skin conditions and additional compatibility profiles for determination of the formulation. For example, the first, second and third ingredients may correspond to a base composition and two booster compositions. Likewise, the first, second, third and fourth ingredients may include a fifth ingredient that includes a base composition and four booster compositions.

The present disclosure includes a method and system where a comprehensive target skin condition diagnosis practice with a targeted and efficacious product formulated and delivered to the user. In one embodiment, a skin diagnosis/questionnaire, using visual aids, is completed to provide targeted skin care system to address the individual user's needs. The users are shown representative images of common skin conditions, where the users view the visual aids and correlate the visual aids with their individual skin care needs. Based on the correlation to the target skin conditions, the users get a customized cosmetic formulation having a base oil composition and at least two selected active booster compositions. In another embodiment, a digital image of a region of skin is analyzed to determine the individual user's skin care needs.

In one embodiment, according to the present disclosure, the skin care composition is dispensed and/or mixed with the cosmetic dispensing system 100. The system includes a base composition dispensing system comprising an aqueous alcohol composition and an aqueous emulsion. The system further includes a booster composition dispensing system comprising each of the following booster compositions: an anti-acne agent; a first grade exfoliating agent; a second grade exfoliating agent; a first grade whitening agent; a second grade whitening agent; a first grade anti-aging agent; and a second grade anti-aging agent. The base composition dispensing system is arranged and disposed to provide one of the base compositions and the booster composition dispensing system arranged and disposed to provide at least two of the booster compositions.

### EXAMPLES

The following examples are intended to further illustrate the present disclosure. They are not intended to limit the disclosure in any way. Unless otherwise indicated, all parts are by weight.

Azelaic acid was dispersed at a concentration of 24% in an oil-free emulsion base to form the azelaic acid booster composition of TABLE 1. The base included methyl methacrylate crosspolymer to provide mattifying skin effects in the final formulation. The 24% azelaic acid dispersion was tested in accelerated stability for 8 weeks. The 24% azelaic acid dispersion was also tested in the final packaging in the dispensing machine to ensure optimal performance.

TABLE 1

| INGREDIENT | CONCENTRATION |
|---|---|
| PHENOXYETHANOL | <1.00 |
| PEG-20 METHYL GLUCOSE | <1.00 |
| SESQUISTEARATE | |
| POLYACRYLATE CROSSPOLYMER-6; | <1.00 |
| XANTHAN GUM | |
| DIMETHICONE (and) DIMETHICONOL | 2.00 |
| DISODIUM EDTA | <1.00 |
| AZELAIC ACID | 24.00 |
| DICAPRYLYL CARBONATE | 2.00 |
| ALCOHOL DENAT.; PROPYLENE GLYCOL, | <20.00 |
| CAPRYLYL GLYCOL | |
| WATER | QS |
| | 100 |

TABLE 2 includes booster compositions utilized in the inventive examples and comparative examples. The amounts of actives in TABLE 2 are the final amounts in a skin care formulation, such as those of Examples 1-36 described below.

TABLE 2

| | Actives in Booster Composition | |
|---|---|---|
| | (final concentration in skin care | |
| Booster # | formulation) | Category |
| 1 | 6.7% glycolic acid, 3.3% lactic acid, | Second Grade Exfoliating agent |
| | 1.7% sodium phytate | |
| 2 | 6% 4-(2-hydroxyethyl)-1- | First Grade Exfoliating agent |
| | piperazineethanesulfonic acid, 5.45% | |
| | hydroxyethyl urea | |
| 3 | 1.8% tranexamic acid, 0.75% urea | Second Grade Whitening Agent |
| 4 | 3% tranexamic acid, 1.25% urea | Second Grade Whitening Agent |
| 5 | 3% niacinamide, 1% kojic acid | Second Grade Whitening Agent |
| 6 | 0.1% licorice extract, 0.0025% | First Grade Whitening Agent |
| | mulberry extract | |
| 7 | 3.5% C-beta-D-xylopyranoside-2- | First Grade Anti-Aging Agent |
| | hydroxypropane | |
| 8 | 0.1% retinol | Second Grade Anti-Aging Agent |
| 9 | 0.3% retinol | Second Grade Anti-Aging Agent |
| 10 | 0.5% retinol | Second Grade Anti-Aging Agent |
| 11 | 0.5% phenylethyl resorcinol | Second Grade Whitening Agent |
| 12 | 5% azelaic acid | Anti-Acne Agent |
| 13 | 12% azelaic acid | Anti-Acne Agent |

The azelaic acid anti-acne boosters, Boosters 12 and 13 in TABLE 2, were stable over a period of 2 years, with a 6-month period-after-opening limit. The booster also performed well in a dispensing system and did not exhibit significant drips or leakage, even after a period of aging.

TABLE 3 includes the composition of Base C, the aqueous emulsion base, utilized in the inventive examples and comparative examples.

TABLE 3
Base C: Aqueous Emulsion Base Composition

| Name | Concentration (% by wt of base composition) |
|---|---|
| disodium EDTA | 0.1 |
| fatty compound | 2 |
| polymer 1 | 0.15 |
| polymer 2 | 0.5 |
| polymer 3 | 0.6 |
| preservative | 0.7 |
| silicon | 2 |
| solvent 1 | 3 |
| solvent 2 | 4 |
| solvent 3 | 3 |
| solvent 4 | 0.3 |
| surfactant | 0.5 |
| vitamin | 0.5 |
| water | 82.65 |

Thirty-six formulations were made as combination of Booster 12 or Booster 13 with two additional Boosters from TABLE 2 and the aqueous emulsion base composition of TABLE 3. Each formulation was tested on accelerated 8-week stability to determine the safety and success for the consumer. Out of the 36 combinations tested, 28 formulas were shown to be stable for three months after the date of manufacture, whereas 8 formulas were not. Mixing studies were conducted on the thickest formulas to determine the correct time for a homogenous mixture. These studies were compared to a standard that was performed previously. These mixing studies also included 8 weeks of visual and microscopic stability tests.

TABLE 4 includes inventive Examples of formulations including combinations of the aqueous emulsion base composition and three booster compositions.

TABLE 4

| EXAMPLE | First Booster | Second Booster | Third Booster | Stability |
|---|---|---|---|---|
| 1 | 2 | 4 | 13 | PASS |
| 2 | 2 | 6 | 13 | PASS |
| 3 | 2 | 7 | 13 | PASS |
| 4 | 2 | 13 | 8 | PASS |
| 5 | 2 | 13 | 9 | PASS |
| 6 | 4 | 6 | 13 | PASS |
| 7 | 4 | 7 | 13 | PASS |
| 8 | 6 | 7 | 13 | PASS |
| 9 | 6 | 13 | 8 | PASS |
| 10 | 6 | 13 | 9 | PASS |
| 11 | 6 | 13 | 10 | PASS |
| 12 | 7 | 13 | 8 | PASS |
| 13 | 7 | 13 | 9 | PASS |
| 14 | 7 | 13 | 10 | PASS |
| 15 | 2 | 3 | 12 | PASS |
| 16 | 2 | 6 | 12 | PASS |
| 17 | 2 | 7 | 12 | PASS |
| 18 | 2 | 12 | 8 | PASS |
| 19 | 2 | 12 | 9 | PASS |
| 20 | 3 | 6 | 12 | PASS |
| 21 | 3 | 7 | 12 | PASS |
| 22 | 6 | 7 | 12 | PASS |
| 23 | 6 | 12 | 8 | PASS |
| 24 | 6 | 12 | 9 | PASS |
| 25 | 6 | 12 | 10 | PASS |
| 26 | 7 | 12 | 8 | PASS |
| 27 | 7 | 12 | 9 | PASS |
| 28 | 7 | 12 | 10 | PASS |

TABLE 5 lists the ingredients and their amounts in weight percentage in inventive Example 7 and inventive Example 21.

TABLE 5

| Ingredient | Example 7 | Example 21 |
|---|---|---|
| DISODIUM EDTA | <1 | <1 |
| PHENOXYETHANOL | <1 | <1 |
| DICAPRYLYL CARBONATE | <2 | <2 |
| TOCOPHEROL | 0.075 | 0.271 |
| TRANEXAMIC ACID | 3.000 | 1.800 |
| UREA | 1.250 | 0.750 |
| 35% HYDROXYPROPYL TETRAHYDROPYRANTRIOL | 10.000 | 10.000 |
| DIMETHICONE (and) DIMETHICONOL | <2 | <2 |
| PEG-20 METHYL GLUCOSE SESQUISTEARATE | <0.4 | <0.4 |
| AZELAIC ACID | 12.000 | 4.999 |
| Polymers/thickening agents/stabilizing agents | <1 | <1 |
| DENATURED ALCOHOL/OTHER SOLVENTS | >12 | >10 |
| WATER | QS | QS |
| | 100 | 100 |

TABLE 6 includes comparative Examples of formulations including combinations of the aqueous emulsion base composition and three booster compositions.

TABLE 6

| EXAMPLE | First Booster | Second Booster | Third Booster | Stability |
|---|---|---|---|---|
| 29 | 2 | 13 | 10 | FAIL |
| 30 | 4 | 8 | 13 | FAIL |
| 31 | 4 | 9 | 13 | FAIL |
| 32 | 4 | 10 | 13 | FAIL |
| 33 | 2 | 12 | 10 | FAIL |
| 34 | 3 | 8 | 12 | FAIL |
| 35 | 3 | 9 | 12 | FAIL |
| 36 | 3 | 10 | 12 | FAIL |

For the comparative Examples, large retinol pooling was observed, typically within 4 to 7 days after forming the comparative Example. In some cases, small crystallization on the edges of the formulation was observed.

In addition to the comparative Examples of TABLE 6, stable formulations combining either Booster 12 or Booster 13 with either Booster 1 or Booster 11 were not produced.

Clinical trials were performed with inventive Example 7 and inventive Example 21.

During the clinical trials, the effect of a facial treatment of inventive Example 7 on thirteen different claims was evaluated over an eight week period. The qualitative results at week 2, week 4, and week 8 for Example 7 are shown in TABLE 7. “X” indicates a statistically significant improvement when compared to the baseline (p≤0.05). “XX” indicates a statistical and clinical significance (minimum change of −0.51) and (p≤0.05) (not applicable for lesion counts).

TABLE 7

| Claims | Assessment | Week 2 | Week 4 | Week 8 |
|---|---|---|---|---|
| Total Inflammatory Lesions | Lesion Count | X | X | X |
| (papules, pustules) | | | | |
| Total Non-Inflammatory Lesions | Lesion Count | | X | X |
| (open comedones, closed | | | | |
| comedones) | | | | |
| PIH/PIE (Post-Inflammatory | Clinical Grading | XX | XX | XX |
| Hyperpigmentation/Post- | | | | |
| Inflammatory Erythema) | | | | |
| Dark Spot Intensity | Clinical Grading | X | XX | XX |
| Dark Spot Size | Clinical Grading | XX | XX | XX |
| Skin Clarity | Clinical Grading | XX | XX | XX |
| Overall Hyperpigmentation | Clinical Grading | XX | XX | XX |
| Fine Lines/Wrinkles | Clinical Grading | X | X | X |
| Uneven Skin Tone | Clinical Grading | XX | XX | XX |
| Rough Skin Texture | Clinical Grading | XX | XX | XX |
| Dullness | Clinical Grading | XX | XX | XX |
| Enlarged Pores | Clinical Grading | X | XX | XX |
| Overall Assessment of Disease | Clinical Grading | XX | XX | XX |

TABLE 8 shows the quantitative results of the clinical evaluation for the first set of clinical trials for inventive Example 7 by an expert grader for product efficacy. A negative value in TABLE 8 represents an improvement for the attribute. A p-value of less than 0.05 indicates statistical significance. A bold value indicates a statistically significant improvement.

TABLE 8

| | | | | | | | | | Mean % | |
|---|---|---|---|---|---|---|---|---|---|---|
| | | | | | | Mean Change | | | Change | |
| | | | | | | from Baseline | | | from | |
| Assessment | Time Point | n | Mean (±SD) | | | (±SD) | | | Baseline | p-value |
| Total Inflammatory | Baseline | 54 | 7.72 | ± | 4.74 | | ± | | | |
| Lesions (papules, | Week 2 | 54 | 4.63 | ± | 5.16 | −3.09 | ± | 2.47 | −40.05% | <0.001 |
| pustules) | Week 4 | 53 | 3.49 | ± | 4.25 | −4.23 | ± | 2.51 | −54.79% | <0.001 |
| | Week 8 | 52 | 3.21 | ± | 4.58 | −4.51 | ± | 2.96 | −58.41% | <0.001 |
| | Week 12 | | | ± | | | ± | | | |
| Total Non- | Baseline | 54 | 7.72 | ± | 6.51 | | ± | | | |
| Inflammatory Lesions | Week 2 | 54 | 7.76 | ± | 7.15 | −0.46 | ± | 2.79 | −6.00% | 0.229 |
| (open comedones, | Week 4 | 53 | 6.08 | ± | 7.23 | −1.65 | ± | 4.41 | −21.32% | 0.009 |
| closed comedones) | Week 8 | 52 | 5.71 | ± | 7.36 | −2.01 | ± | 4.47 | −26.03% | 0.002 |
| | Week 12 | | | ± | | | ± | | | |
| PIH/PIE | Baseline | 54 | 2.46 | ± | 0.50 | | ± | | | |
| (Post-Inflammatory | Week 2 | 54 | 1.80 | ± | 0.56 | −0.67 | ± | 0.58 | −27.07% | <0.001 |
| Hyperpigmentation/ | Week 4 | 53 | 1.68 | ± | 0.67 | −0.78 | ± | 0.72 | −31.82% | <0.001 |
| Post-Inflammatory | Week 8 | 52 | 1.83 | ± | 0.68 | −0.64 | ± | 0.76 | −25.82% | <0.001 |
| Erythema) | Week 12 | | | ± | | | ± | | | |
| Dark Spot Intensity | Baseline | 54 | 2.17 | ± | 0.38 | | ± | | | |
| | Week 2 | 54 | 1.67 | ± | 0.58 | −0.50 | ± | 0.61 | −23.08% | <0.001 |
| | Week 4 | 53 | 1.58 | ± | 0.69 | −0.57 | ± | 0.70 | −26.85% | <0.001 |
| | Week 8 | 52 | 1.56 | ± | 0.67 | −0.61 | ± | 0.72 | −28.10% | <0.001 |
| | Week 12 | | | ± | | | ± | | | |
| Dark Spot Size | Baseline | 54 | 2.15 | ± | 0.36 | | ± | | | |
| | Week 2 | 54 | 1.63 | ± | 0.56 | −0.52 | ± | 0.61 | −24.14% | <0.001 |
| | Week 4 | 53 | 1.40 | ± | 0.66 | −0.75 | ± | 0.62 | −35.00% | <0.001 |
| | Week 8 | 52 | 1.44 | ± | 0.64 | −0.71 | ± | 0.64 | −32.85% | <0.001 |
| | Week 12 | | | ± | | | ± | | | |
| Skin Clarity | Baseline | 54 | 2.56 | ± | 0.50 | | ± | | | |
| | Week 2 | 54 | 1.69 | ± | 0.64 | −0.87 | ± | 0.62 | −34.06% | <0.001 |
| | Week 4 | 53 | 1.64 | ± | 0.65 | −0.91 | ± | 0.63 | −35.76% | <0.001 |
| | Week 8 | 52 | 1.48 | ± | 0.70 | −1.07 | ± | 0.62 | −42.05% | <0.001 |
| | Week 12 | | | ± | | | ± | | | |
| Overall | Baseline | 54 | 2.26 | ± | 0.44 | | ± | | | |
| Hyperpigmentation | Week 2 | 54 | 1.63 | ± | 0.62 | −0.63 | ± | 0.62 | −27.87% | <0.001 |
| | Week 4 | 53 | 1.64 | ± | 0.59 | −0.62 | ± | 0.53 | −27.34% | <0.001 |
| | Week 8 | 52 | 1.58 | ± | 0.64 | −0.68 | ± | 0.62 | −30.20% | <0.001 |
| | Week 12 | | | ± | | | ± | | | |
| Fine Lines/Wrinkles | Baseline | 54 | 2.13 | ± | 0.34 | | ± | | | |
| | Week 2 | 54 | 1.85 | ± | 0.45 | −0.28 | ± | 0.45 | −13.04% | <0.001 |
| | Week 4 | 53 | 1.96 | ± | 0.39 | −0.17 | ± | 0.38 | −7.89% | 0.004 |
| | Week 8 | 52 | 1.94 | ± | 0.37 | −0.19 | ± | 0.44 | −8.79% | 0.004 |
| | Week 12 | | | ± | | | ± | | | |
| Uneven Skin Tone | Baseline | 54 | 2.30 | ± | 0.46 | | ± | | | |
| | Week 2 | 54 | 1.61 | ± | 0.68 | −0.69 | ± | 0.64 | −29.84% | <0.001 |
| | Week 4 | 53 | 1.74 | ± | 0.56 | −0.56 | ± | 0.57 | −24.40% | <0.001 |
| | Week 8 | 52 | 1.63 | ± | 0.63 | −0.66 | ± | 0.65 | −28.81% | <0.001 |
| | Week 12 | | | ± | | | ± | | | |
| Rough Skin Texture | Baseline | 54 | 2.24 | ± | 0.43 | | ± | | | |
| | Week 2 | 54 | 1.54 | ± | 0.66 | −0.70 | ± | 0.63 | −31.40% | <0.001 |
| | Week 4 | 53 | 1.34 | ± | 0.62 | −0.90 | ± | 0.64 | −40.21% | <0.001 |
| | Week 8 | 52 | 1.31 | ± | 0.51 | −0.93 | ± | 0.55 | −41.64% | <0.001 |
| | Week 12 | | | ± | | | ± | | | |
| Dullness | Baseline | 54 | 2.31 | ± | 0.47 | | ± | | | |
| | Week 2 | 54 | 1.63 | ± | 0.56 | −0.69 | ± | 0.54 | −29.60% | <0.001 |
| | Week 4 | 53 | 1.08 | ± | 0.43 | −1.24 | ± | 0.61 | −53.53% | <0.001 |
| | Week 8 | 52 | 1.12 | ± | 0.38 | −1.20 | ± | 0.60 | −51.81% | <0.001 |
| | Week 12 | | | ± | | | ± | | | |
| Enlarged Pores | Baseline | 54 | 2.30 | ± | 0.46 | | ± | | | |
| | Week 2 | 54 | 1.81 | ± | 0.48 | −0.48 | ± | 0.54 | −20.97% | <0.001 |
| | Week 4 | 53 | 1.60 | ± | 0.53 | −0.69 | ± | 0.73 | −30.15% | <0.001 |
| | Week 8 | 52 | 1.62 | ± | 0.49 | −0.68 | ± | 0.62 | −29.65% | <0.001 |
| | Week 12 | | | ± | | | ± | | | |
| Overall Assessment | Baseline | 54 | 2.39 | ± | 0.49 | | ± | | | |
| of Disease | Week 2 | 54 | 1.83 | ± | 0.54 | −0.56 | ± | 0.54 | −23.26% | <0.001 |
| | Week 4 | 53 | 1.75 | ± | 0.48 | −0.63 | ± | 0.56 | −26.54% | <0.001 |
| | Week 8 | 52 | 1.60 | ± | 0.57 | −0.79 | ± | 0.64 | −33.18% | <0.001 |
| | Week 12 | | | ± | | | ± | | | |

In summary, a statistically significant improvement in total inflammatory lesions was observed after just two weeks of Example 7 use, and a statistically significant improvement in total non-inflammatory lesions was observed after just four weeks of Example 7 use. A statistically and clinically significant improvement in PIH/PIE, dark spot size, skin clarity, overall hyperpigmentation, uneven skin tone, rough skin texture, dullness, and overall assessment of disease was observed after 2, 4, and 8 weeks of Example 7 use. A statistically and clinically significant improvement in dark spot intensity and enlarged pores was observed after 4 and 8 weeks of Example 7 use.

The effect of a facial treatment of inventive Example 21 on the same thirteen claims was evaluated over a twelve week period. The results at week 2, week 4, and week 12 for Example 21 are shown in TABLE 9.

TABLE 9

| Claims | Assessment | Week 2 | Week 4 | Week 12 |
|---|---|---|---|---|
| Total Inflammatory Lesions | Lesion Count | X | X | X |
| (papules, pustules) | | | | |
| Total Non-Inflammatory Lesions | Lesion Count | | X | |
| (open comedones, closed | | | | |
| comedones) | | | | |
| PIH/PIE (Post-Inflammatory | Clinical Grading | X | XX | XX |
| Hyperpigmentation/Post- | | | | |
| Inflammatory Erythema) | | | | |
| Dark Spot Intensity | Clinical Grading | X | X | XX |
| Dark Spot Size | Clinical Grading | X | X | XX |
| Skin Clarity | Clinical Grading | X | XX | XX |
| Overall Hyperpigmentation | Clinical Grading | X | X | XX |
| Fine Lines/Wrinkles | Clinical Grading | X | X | X |
| Uneven Skin Tone | Clinical Grading | X | X | XX |
| Rough Skin Texture | Clinical Grading | XX | XX | XX |
| Dullness | Clinical Grading | XX | XX | XX |
| Enlarged Pores | Clinical Grading | X | X | |
| Overall Assessment of Disease | Clinical Grading | X | X | |

TABLE 10 shows the quantitative results of the clinical evaluation for the first set of clinical trials for inventive Example 21 by an expert grader for product efficacy. A negative value in TABLE 10 represents an improvement for the attribute. A p-value of less than 0.05 indicates statistical significance. A bold value indicates a statistically significant improvement.

TABLE 10

| | | | | | | | | | Mean % | |
|---|---|---|---|---|---|---|---|---|---|---|
| | | | | | | Mean Change | | | Change | |
| | | | | | | from Baseline | | | from | |
| Assessment | Time Point | n | Mean (±SD) | | | (±SD) | | | Baseline | p-value |
| Total Inflammatory | Baseline | 50 | 6.24 | ± | 3.25 | | | | | |
| Lesions (papules, | Week 2 | 50 | 4.58 | ± | 3.89 | −1.76 | ± | 2.38 | −27.83% | <0.001 |
| pustules) | Week 4 | 47 | 4.19 | ± | 5.48 | −2.13 | ± | 3.96 | −33.69% | 0.002 |
| | Week 8 | | | | | | | | | |
| | Week 12 | 50 | 3.84 | ± | 5.55 | −2.47 | ± | 3.38 | −39.19% | 0.010 |
| Total Non- | Baseline | 50 | 8.02 | ± | 6.47 | | | | | |
| Inflammatory | Week 2 | 50 | 7.20 | ± | 7.60 | −0.64 | ± | 3.99 | −8.13% | 0.159 |
| Lesions (open | Week 4 | 47 | 6.28 | ± | 7.63 | −1.64 | ± | 4.35 | −20.85% | 0.011 |
| comedones, closed | Week 8 | | | | | | | | | |
| comedones) | Week 12 | 50 | 5.72 | ± | 7.60 | −2.15 | ± | 4.66 | −27.32% | 0.106 |
| PIH/PIE | Baseline | 50 | 2.46 | ± | 0.50 | | | | | |
| (Post-Inflammatory | Week 2 | 50 | 2.13 | ± | 0.62 | −0.31 | ± | 0.64 | −12.67% | 0.004 |
| Hyperpigmentation/ | Week 4 | 47 | 1.91 | ± | 0.65 | −0.55 | ± | 0.62 | −22.44% | <.001 |
| Post-Inflammatory | Week 8 | | | | | | | | | |
| Erythema) | Week 12 | 50 | 1.60 | ± | 0.73 | −0.84 | | 0.73 | −34.54% | <.001 |
| Dark Spot Intensity | Baseline | 50 | 2.28 | ± | 0.45 | | | | | |
| | Week 2 | 50 | 2.10 | ± | 0.51 | −0.18 | ± | 0.58 | −7.97% | 0.058 |
| | Week 4 | 47 | 1.91 | ± | 0.54 | −0.36 | ± | 0.57 | −15.85% | <.001 |
| | Week 8 | | | | | | | | | |
| | Week 12 | 50 | 1.46 | ± | 0.65 | −0.82 | | 0.72 | −35.90% | <.001 |
| Dark Spot Size | Baseline | 50 | 2.32 | ± | 0.47 | | | | | |
| | Week 2 | 50 | 2.10 | ± | 0.51 | −0.24 | ± | 0.55 | −10.27% | 0.011 |
| | Week 4 | 47 | 1.91 | ± | 0.58 | −0.42 | ± | 0.68 | −18.10% | <.001 |
| | Week 8 | | | | | | | | | |
| | Week 12 | 50 | 1.40 | ± | 0.57 | −0.91 | | 0.75 | −39.52% | <.001 |
| Skin Clarity | Baseline | 50 | 2.38 | ± | 0.49 | | | | | |
| | Week 2 | 50 | 1.94 | ± | 0.65 | −0.45 | ± | 0.67 | −18.87% | <.001 |
| | Week 4 | 47 | 1.66 | ± | 0.76 | −0.70 | ± | 0.74 | −29.68% | <.001 |
| | Week 8 | | | | | | | | | |
| | Week 12 | 50 | 1.18 | ± | 0.98 | −1.19 | | 0.99 | −50.21% | <.001 |
| Overall | Baseline | 50 | 2.24 | ± | 0.43 | | | | | |
| Hyperpigmentation | Week 2 | 50 | 2.10 | ± | 0.46 | −0.16 | ± | 0.50 | −7.31% | 0.055 |
| | Week 4 | 47 | 2.00 | ± | 0.51 | −0.26 | ± | 0.54 | −11.67% | 0.005 |
| | Week 8 | | | | | | | | | |
| | Week 12 | 50 | 1.54 | ± | 0.71 | −0.70 | | 0.81 | −31.27% | <.001 |
| Fine Lines/Wrinkles | Baseline | 50 | 2.12 | ± | 0.32 | | | | | |
| | Week 2 | 50 | 2.02 | ± | 0.31 | −0.09 | ± | 0.36 | −4.35% | 0.109 |
| | Week 4 | 47 | 1.94 | ± | 0.32 | −0.17 | ± | 0.39 | −8.22% | 0.004 |
| | Week 8 | | | | | | | | | |
| | Week 12 | 50 | 1.80 | ± | 0.45 | −0.31 | | 0.47 | −14.73% | <.001 |
| Uneven Skin Tone | Baseline | 50 | 2.36 | ± | 0.48 | | | | | |
| | Week 2 | 50 | 2.06 | ± | 0.51 | −0.29 | ± | 0.67 | −12.50% | 0.008 |
| | Week 4 | 47 | 1.89 | ± | 0.70 | −0.46 | ± | 0.80 | −19.38% | 0.002 |
| | Week 8 | | | | | | | | | |
| | Week 12 | 50 | 1.54 | ± | 0.65 | −0.81 | | 0.72 | −34.51% | <.001 |
| Rough Skin Texture | Baseline | 50 | 2.32 | ± | 0.47 | | | | | |
| | Week 2 | 50 | 1.72 | ± | 0.61 | −0.62 | ± | 0.66 | −26.89% | <.001 |
| | Week 4 | 47 | 1.43 | ± | 0.71 | −0.88 | ± | 0.70 | −37.90% | <.001 |
| | Week 8 | | | | | | | | | |
| | Week 12 | 50 | 1.32 | | 0.71 | −0.99 | | 0.64 | −42.97% | <.001 |
| Dullness | Baseline | 50 | 2.32 | ± | 0.47 | | | | | |
| | Week 2 | 50 | 1.30 | ± | 0.51 | −1.03 | ± | 0.58 | −44.33% | <.001 |
| | Week 4 | 47 | 1.30 | ± | 0.59 | −1.02 | ± | 0.67 | −44.20% | <.001 |
| | Week 8 | | | | | | | | | |
| | Week 12 | 50 | 0.96 | ± | 0.75 | −1.35 | | 0.80 | −58.52% | <.001 |
| Enlarged Pores | Baseline | 50 | 2.18 | ± | 0.52 | | | | | |
| | Week 2 | 50 | 1.86 | ± | 0.50 | −0.32 | ± | 0.67 | −14.63% | 0.005 |
| | Week 4 | 47 | 1.70 | ± | 0.59 | −0.48 | ± | 0.74 | −21.82% | <.001 |
| | Week 8 | | | | | | | | | |
| | Week 12 | 50 | 1.60 | ± | 0.61 | −0.59 | | 0.70 | −26.77% | <.001 |
| Overall Assessment | Baseline | 50 | 2.32 | ± | 0.62 | | | | | |
| of Disease | Week 2 | 50 | 2.14 | ± | 0.45 | −0.18 | ± | 0.58 | −7.78% | 0.058 |
| | Week 4 | 47 | 1.96 | ± | 0.55 | −0.36 | ± | 0.73 | −15.40% | 0.005 |
| | Week 8 | | | | | | | | | |
| | Week 12 | 50 | 1.58 | ± | 0.61 | −0.73 | | 0.75 | −31.74% | <.001 |

In summary, a statistically significant improvement in total inflammatory lesions, rough skin texture, and dullness was observed after just two weeks of Example 21 use, a statistically significant improvement in total non-inflammatory lesions, PIE/PIE, and skin clarity was observed after just four weeks of Example 21 use, and a statistically significant improvement in dark spot size, dark spot intensity, overall hyperpigmentation, skin tone evenness, enlarged pores, and overall skin appearance was observed after twelve weeks of Example 21 use.

During the clinical trials, the cutaneous acceptability of Example 7 and Example 21 was clinically evaluated by a dermatologist in terms of peeling, dryness, redness, and swelling, and all criteria were determined to be stable at all of the observed time points indicated in TABLE 8 and TABLE 10.

During the clinical trials, the cutaneous acceptability of Example 21 was assessed by the subject in terms of itching, stinging, redness, and swelling at all of the observed time points indicated in TABLE 10. All criteria were determined to be stable except for itching at week two and week four, stinging at week two, and redness at week two, week four, and week twelve.

While the disclosure has been described with reference to a preferred embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

The terms “free” and “devoid” indicates that no reliably measurable excluded material is present in the composition, typically 0% by weight, based on the total weight of the composition. The term “essentially free” means that, while it prefers that no excluded material is present in the composition, it is possible to have very small amounts of the excluded material in the composition of the invention, provided that these amounts do not materially affect the advantageous properties of the composition. In particular, “essentially free” means that excluded material can be present in the composition at an amount of less than about 0.1% by weight, based on the total weight of the composition.

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The examples serve to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

What is claimed is:

1. A skin care formulation comprising:
1-20% by weight azelaic acid;
a first booster composition comprising at least one first active booster ingredient;
a second booster composition comprising at least one second active booster ingredient;
wherein the skin care formulation is a water-based emulsion; and
wherein the water-based emulsion is stable for at least 3 months.

2. The skin care formulation of claim 1 further comprising an aqueous emulsion base composition.

3. The skin care formulation of claim 1 further comprising at least one glycol at a concentration of at least 2% by weight of the skin care formulation.

4. The skin care formulation of claim 1, wherein the skin care formulation comprises 3-15% by weight azelaic acid.

5. The skin care formulation of claim 1, wherein the skin care formulation is essentially free of one or more of: alcohols, oils other than silicone oils, and combinations thereof.

6. The skin care formulation of claim 1, wherein the at least one first active booster ingredient and the at least one second active booster ingredient are independently selected from the group consisting of a first grade exfoliating agent, a second grade exfoliating agent, a first grade whitening agent, a second grade whitening agent, a first grade anti-aging agent, and a second grade anti-agent agent.

7. The skin care formulation of claim 1, wherein the at least one first active booster ingredient and the at least one second active booster ingredient are not selected from the group consisting of phenylethyl resorcinol and a combination of glycolic acid, lactic acid, and sodium phytate.

8. A method of forming a skin care formulation comprising:
combining an anti-acne booster composition, a first booster composition, a second booster composition, and an aqueous base composition to form the skin care formulation;
wherein the anti-acne booster composition comprises azelaic acid;
wherein the first booster composition comprises at least one first active booster ingredient;
wherein the second booster composition comprises at least one second active booster ingredient;
wherein the skin care formulation comprises azelaic acid;
wherein the skin care formulation is a water-based emulsion; and
wherein the water-based emulsion is stable for at least 3 months.

9. The method of claim 8, wherein the anti-acne booster composition comprises at least 20% azelaic acid by weight of the anti-acne booster composition and wherein the anti-acne booster composition is stable for at least 6 months.

10. The method of claim 8, wherein the anti-acne booster composition further comprising at least 50% water, less than 5% alcohol, and at least 2% of at least one glycol by weight of the anti-acne booster composition.

11. The method of claim 8, wherein the anti-acne booster composition is essentially free of one or more of: alcohols, oils other than silicone oils, and combinations thereof.

12. The method of claim 8, wherein the skin care formulation comprises 3-15% by weight azelaic acid.

13. The method of claim 8 further comprising independently selecting the at least one first active booster ingredient and the at least one second active booster ingredient from the group consisting of a first grade exfoliating agent, a second grade exfoliating agent, a first grade whitening agent, a second grade whitening agent, a first grade anti-aging agent, and a second grade anti-agent agent, based on a skin condition of a consumer.

14. The method of claim 8 further comprising not selecting the at least one first active booster ingredient and the at least one second active booster ingredient from the group consisting of phenylethyl resorcinol and a combination of glycolic acid, lactic acid, and sodium phytate.

15. The method of claim 8 further comprising selecting the anti-acne booster composition, the first booster composition, and the second booster composition based on a skin condition of a consumer.

16. A method for managing a skin care system for providing a customized skin care product to a user, the skin care system including one or more input/output devices configured to display information to a user and receive input from the user, one or more cosmetic dispensing systems configured to dispense and mix a cosmetic composition, and a skin care management system, coupled to the one or more input/output devices and the one or more cosmetic dispensing systems, and including a microprocessor and a memory, the method comprising:
displaying, by the skin care management system, one or more visual skin guides to the user, each of the visual skin guides corresponding to one or more skin conditions including one or more visual representations of the one or more skin conditions;
receiving, by the skin care management system, one or more inputs from the user including skin information corresponding to the one or more visual representations of the one or more skin conditions of the one or more visual skin guides;
determining, by the skin care management system, one or more skin conditions based on the one or more inputs from the user;
determining, by the skin care management system, one or more severity scores based on the one or more skin conditions;
determining, by the skin care management system, a primary skin condition based on the severity scores;
selecting, by the skin care management system, a first ingredient based on the one or more severity scores;
generating, by the skin care management system, a compatibility profile based on the first ingredient;
determining, by the skin care management system, one or more secondary skin conditions based on the one or more severity scores;
selecting, by the skin care management system, an additional ingredient based on the one or more severity scores;
updating, by the skin care management system, the compatibility profile based on the additional ingredient;
displaying, by the skin care management system, one or more cosmetic formulations to the user;
receiving, by the skin care management system, a selection of one or more cosmetic formulations from the user;
wherein, at least one of the determined skin conditions comprises acne and one of the generated ingredients is an anti-acne booster composition comprising azelaic acid; and
wherein the cosmetic composition to be dispensed and mixed by the skin care management system comprises at least four ingredients comprising a base composition, an anti-acne booster composition comprising azelaic acid, and at least two additional ingredients comprising at least one first active booster composition and one second active booster composition, the at least one first active booster composition and the at least one second active booster composition each selected from the group consisting of a first grade exfoliating agent, a second grade exfoliating agent, a first grade whitening agent, a second grade whitening agent, a first grade anti-aging agent, and a second grade anti-agent agent, based on the at least one skin condition of the consumer.

17. The method of claim 16, wherein the anti-acne booster composition comprises at least 20% azelaic acid by weight of the anti-acne booster composition and wherein the anti-acne booster composition is stable for at least 6 months.

18. The method of claim 16, further comprising regulating, by the skin care management system, the dispensing, by the one or more cosmetic dispensing systems, of the one or more cosmetic formulations, and the mixing, by the one or more cosmetic dispensing systems, of the one or more cosmetic formulations.

19. The method of claim 16 further comprising not selecting the at least one first active booster ingredient and the at least one second active booster ingredient from the group consisting of phenylethyl resorcinol and a combination of glycolic acid, lactic acid, and sodium phytate.

20. The method of claim 16, wherein the cosmetic composition to be dispensed and mixed by the skin care management system comprises 3-15% by weight azelaic acid.
